SEQUENCE LISTING (10 sequences)

SEQ ID NO: 1 - DNA, 59 bp, artificial sequence
  synthesized
gaaggcgatcgctctagagagctggtttcctctttaggtgagctcactagagcgatcgc

SEQ ID NO: 2 - DNA, 59 bp, artificial sequence
  synthesized
tcccagggcccactgcagggatcggtttctcttttaggtggatccatgcagtgggccct

SEQ ID NO: 3 - DNA, 90 bp, artificial sequence
  synthesized
tcccggtgctctcgcgacagctgttgatcaatgtttttgtgtttctgagtcttcagactttgcaatatgcagagctgtcgcgagagcacc

SEQ ID NO: 4 - DNA, 49 bp, artificial sequence
  synthesized
cagctgctcgagtctagagttaactttgcagctagactcgagcagctgt

SEQ ID NO: 5 - DNA, 26 bp, artificial sequence
  synthesized
ggtctctgggatatcggtctctgaag

SEQ ID NO: 6 - DNA, 26 bp, artificial sequence
  synthesized
cttcagagaccgatatcccagagacc

SEQ ID NO: 7 - DNA, 67 bp, artificial sequence
  synthesized
ggtctctgggatatccaagctttaaatggttatagccatggaactagtggatcccgggtctctgaag

SEQ ID NO: 8 - DNA, 67 bp, artificial sequence
  synthesized
cttcagagacccgggatccactagttccatggctataaccatttaaagcttggatatcccagagacc

SEQ ID NO: 9 - DNA, 25 bp, artificial sequence
  synthesized
aagcttcgggcaattgctgtgccag

SEQ ID NO: 10 - DNA, 20 bp, artificial sequence
  synthesized
cggcaataacatacggcgtg

(19) United States
(12) Patent Application Publication
(10) Pub. No.: US 2011/0196142 A1
(43) Pub. Date: Aug. 11, 2011

(54) DNA molecule for expressing Hairpin RNA, the constructing method and the use thereof

(75) Inventors: Lei Wang, Beijing (CN); YunLiu Fan, Beijing (CN)

(73) Assignee: Biotechnology Research Institute The Chinese Academy of Agricultural Sciences, Haidian District, Beijing (CN)

(21) Appl. No.: 13/062,178

(22) PCT Filed: Sep. 4, 2008
(86) PCT No.: PCT/CN2008/001576

§ 371 Date: Mar. 3, 2011

(51) Int. Cl. C07H 21/04; C12P 19/34

(52) U.S. Cl. 536/23.1; 435/91.53

(57) ABSTRACT

DNA molecule for expressing hairpin RNA, the constructing method and the use thereof are provided. The DNA molecule is a closed single-strand cyclic DNA molecule which is constructed by linking four fragments A, B, C and D as following order: A-C-B-D or A-D-B-C, wherein A is a sense strand or anti-sense strand fragment of the target double-strand DNA; B is the fragment which is reversely complementary to A; C and D are DNA fragments with stem-loop structure and they meet one of the following requirements: 1) the nucleotide sequence of fragment C or D comprises at least one sequence e; 2) the nucleotide sequence of fragment C comprises at least one sequence e, the nucleotide sequence of fragment D comprises at least one sequence f, wherein sequences e and f individually are one strand of different restriction endonuclease recognition sequences. The DNA molecule for expressing hairpin RNA and its constructing method are useful in constructing lhRNA library.

### FIELD OF THE INVENTION

The invention relates to a DNA molecule for expressing hairpin RNA, a constructing method and use thereof.

### BACKGROUND OF THE INVENTION

RNA-mediated gene silencing/RNA interfering (RNAi) can induce the degradation of the specific homologous gene mRNA, is an effective tool for inhibiting the gene expression in eukaryotes and is widely used in the identification of gene function and the genetic engineering. By using hairpin RNA (hpRNA), the gene silencing efficiency can generally reach over 70%, which is much higher than that obtained with the antisense RNA strategy. Therefore, hpRNA is the most widely used technology for gene silencing in animals, plants and microorganisms (Wesley, S. V., Plant J. 27, 581-590).

There are a variety of methods for constructing hpRNA at present, the most commonly used is two-step method, i.e. a target sequence is inserted into a vector, then the sequence is inversely inserted once again to form a inverted repeat structure, which can transcribe the hpRNA. The target sequences are inserted into a vector in a reverse orientation using the recombinase by twice recombination in the Gateway recombinant technology (Tomari, Y. Genes Dev 19, 517-529). Recently artificially synthesized miRNA (micro RNA) also can be specifically silencing target genes (Miki, D. Plant Cell Physiol 45, 490-495). However, these methods can only manipulate one DNA fragment at a time, which is suitable for constructing one or a few target gene silencing vectors, is time ineffective and hard sledding, and is unable to construct the gene silencing library.

In recent years, the short hairpin RNA (shRNA) library is constructed by short DNA fragment in animals. Luo et. al. constructed shRNA expression library using SPEED (siRNA production by enzymatic engineering of DNA) strategy (Luo, B. Proc Natl Acad Sci USA, 101, 5494-5499). Shirane et. al. also constructed shRNA expression library using REGS (restriction enzyme-generated siRNA) system (Shirane, D., Nat Genet. 36, 190-196). Sen et. al. constructed shRNA expression library utilizing EPRIL (enzymatic production of RNAi library) technology (Sen, G., Wehrman, Nat Genet. 36, 183-189). However, these technologies adopt similar methods, they all utilize the property of the restriction endonuclease MmeI, and the lengths of the stems in the siRNA library or the shRNA library constructed are all about 20 bp. This short hairpin structure RNA (shRNA) is not only low silencing efficiency, but also poor silencing effect compared to the long hairpin RNA (lhRNA) with long stems (such as above 40 bp). Thus how to construct a hairpin RNA (lhRNA) library with long stems is a challenge at present. There is no any report about the construction of a hairpin RNA library with the stem length above 40 bp.

### DESCRIPTION OF THE INVENTION

An object of the invention is to provide a DNA molecule for expressing hairpin RNA, a constructing method and use thereof.

The DNA molecule for expressing hairpin of the invention is a closed single-strand cyclic molecule consisting of four single-strand DNA fragments A, B, C and D; and said four fragments A, B, C and D are linked in the order of 1) or 2) as follows:

1) A-C-B-D;

2) A-D-B-C;

A is a fragment selected from a sense or a antisense strand of the target double-strand DNA fragment, and the size of said A is 40-3000 nt;

B is a fragment which is reversely complementary to A;

C and D are DNA fragments with the stem-loop structure, and said C and D fragments meet the requirement of 3) or 4) as follows:

3) The nucleotide sequence of said fragment C or D comprises at least one sequence e, said sequence e forms a recognition sequence R of restriction endonuclease with the complementary sequence thereof;

4) The nucleotide sequence of said fragment C comprises at least one sequence e, said sequence e forms a recognition sequence R of restriction endonuclease with the complementary sequence thereof; the nucleotide sequence of said fragment D comprises at least one sequence f, said sequence f forms a recognition sequence S of restriction endonuclease with the complementary sequence thereof; said recognition sequences R and S are recognized by the different restriction endonucleases.

Wherein, the size of said A also can be 100-2000 nt, 100-1000 nt, 200-800 nt or 200-600 nt. The nucleotide sequences of said C and D can be same, such as SEQ ID NO:4 in the sequence list, or different, without any other restriction, for example the sequences are SEQ ID NO:1 and SEQ ID NO:2 in the sequence list respectively. The loop of the stem-loop structure of said C or D also can comprise an intron sequence or not, the length can be designed according to the requirement, it may be long or short. For example, SEQ ID NO:3 in the sequence list provides a stem-loop structure whose loop comprises a intron sequence. The DNA fragments of said C and D may be obtained by using various methods in the art, such as artificial synthesis or single-prime PCR.

Another object of the invention is to provide a constructing method for the DNA molecule for expressing the hairpin RNA.

The constructing method for the DNA molecule for expressing the hairpin RNA of the invention comprises linking three O, P and Q DNA fragments into a closed single-strand cyclic molecule.

Said fragment O is a target double-strand DNA fragment with two cohesive ends which can not be self linked.

Said fragments P and Q are DNA fragments with stem-loop structure having cohesive ends respectively, the cohesive ends of said fragments P and Q are matched with the two cohesive ends of said fragment O, and said fragments P and Q meet the requirement of i) or ii) as follows:

i) the nucleotide sequence of fragment P or Q comprises at least one sequence e, said sequence e forms a recognition sequence R of restriction endonuclease with the complemented sequence thereof;

ii) the nucleotide sequence of said fragment P comprises at least one sequence e, said sequence e forms a recognition sequence R of restriction endonuclease with the complemented sequence thereof; the nucleotide sequence of said fragment Q comprises at least one sequence f, said sequence f forms a recognition sequence S of restriction endonuclease with the complemented sequence thereof; said recognition sequence R and S are recognized by the different restriction endonucleases.

Said fragments P and Q are DNA fragments with stem-loop structure having cohesive ends respectively, the cohesive ends of said fragments P and Q are matched with the two cohesive ends of said fragment O, this ensures that the cohesive ends of said fragment P or Q can not be self linked and they can not be linked into P-P, Q-Q and P-Q closed single-strand cyclic DNA in the enzyme-chain reaction.

Wherein, said three DNA fragments O, P and Q may be obtained by using various methods in the art, such as artificially synthesis or cloning. said fragment P and Q of the invention are artificially synthesized. The length of the projecting cohesive end of said fragment O can be above 1 nt, it also can be in the range of 1-9 nt or 1-20 nt. said fragment O can be obtained through PCR amplification or PCR product enzyme cleavage (a specific endonuclease enzyme cleavage site is introduced into PCR product) or linking the specific linkers and the like. For example, two overhung cohesive ends of the fragment O are A, and the length is 1 nt.

Said fragment O of the invention can be made using any of the three methods as follows:

The first method comprises the steps as follows:

1) The exogenous fragment is inserted into the poly clone sites of a gene cloning vector or gene expression vector to obtain a recombinant vector M; the nucleotide sequence of said exogenous fragment contains three recognition sequences a, b and c of the restriction endonuclease, said sequences a and c can produce unself-linked cohesive ends after the restriction endonuclease enzyme cleavage; the linking order of said three recognition sequence is a-b-c;

2) Said target double-strand DNA fragment is inserted into said b site of said recombinant vector M to get a recombinant vector N;

3) The recombinant vector N is enzymatically cleaved by the restrictive endonuclease recognizing said sequence a and c to get said fragment O.

Wherein, said gene cloning vector or gene expression vector may choice various vectors, such as pUC18, pUC19, pBlueScript, pQE, pGEX and pET and the like.

The second method comprises the steps as follows:

1) The exogenous fragment is inserted into the multiple cloning site in the gene cloning vector or gene expression vector to obtain a recombinant vector M; the nucleotide sequence of said exogenous fragment contains two recognition sequences a and c of the restriction endonuclease, said sequences a and c produce the unself-linked cohesive ends after the restriction endonuclease enzyme cleavage; there is a fragment b between said a and c, said fragment b contains two restriction endonuclease recognition sequences b-1 and b-2, two free basic bases of two overhung ends produced after the restriction endonuclease enzyme cleavage said sequences b-1 and b-2 are T;

2) The recombinant vector M is enzymatically cleaved by the restriction endonuclease recognizing said b-1 and b-2 to obtain a recombinant vector T;

3) The target double-strand DNA fragment is prepared, and two ends of the double-strand DNA fragment are overhung ends with free basic bases A;

4) The recombinant vector T of step 2) and the double-strand DNA fragment of step 3) are linked to obtain a recombinant vector N;

5) The recombinant vector N is enzymatically cleaved by the restriction endonuclease recognizing said sequences a and c to obtain said fragment O.

Wherein, said gene cloning vector or gene expression vector may be selected from various vectors, such as pUC18, pUC19, pBlueScript, pQE, pGEX and pET and the like. said recognition sequences b-1 and b-1 may be same or different. The restriction endonucleases recognizing said b-1 and b-2 are some restriction endonucleases which can be directly obtained and have cohesive ends “T”, such as Ahd I, BciV I, Bmr I, Xcm I and the like.

The third method comprises the steps as follows:

1) The exogenous fragment is inserted into the multiple cloning site of a gene cloning vector or gene expression vector to obtain a recombinant vector M; the nucleotide sequence of said exogenous fragment contains three recognition sequences a, b and c of the restriction endonuclease, said sequences a and c produce the unself-linked cohesive ends after the restriction endonuclease enzyme cleavage; the order of linking said three recognition sequences is a-b-c;

2) The recombinant vector M is enzymatically cleaved by the restriction endonuclease recognizing said sequence b to prepare vector T;

3) The target double-strand DNA fragment is prepared, and two ends of the double-strand DNA fragment are overhung ends with free basic bases A;

4) The vector T of step 2) and the double-strand DNA fragment of step 3) are linked to obtain a recombinant vector N;

5) The recombinant vector N is enzymatically cleaved by the restriction endonucleases recognizing said sequences a and c to obtain said fragment O.

Wherein, said gene cloning vector or gene expression vector may be selected from various vectors, such as pUC18, pUC19, pBlueScript, pQE, pGEX and pET and the like. To avoid the vector to self link and to facilitate the linkage of PCT products, the sequence produces planar ends after enzymatically cleaved by the restriction endonuclease recognizing said b, then adding one “T” at 3′ end by Taq enzyme to form T vector; alternatively, the sequence produces cohesive ends after enzymatically cleaved by the restriction endonuclease recognizing said b, the ends are made up to planar ends, then adding one “T” at 3′ end by Taq enzyme to form T vector.

In these three methods above, the cohesive ends of said sequences a and c produced through restriction endonuclease enzyme cleavage are unself-linked cohesive ends, the restriction endonuclease may be selected from various commercial endonucleases, such as, Afl III, Ahd I, Alw I, AlwN, Ava I, Ban I, Ban II, Bbs I, Bbv I, BciV I, Bgl I, Blp I, Bmr I, Bsa I, BseR I, Bsm I, BsmA I, BsmB I, BsoB I, BspM I, Bsr I, BsrD I, BstAP I, BstE II, BstF5 I, BstX I, Bsu36 I, Btg I, Bts I, Dra III, Drd I, Ear I, Eci I, EcoN I, EcoO109 I, Hga I, Hph I, Mbo II, PflF I, PflM I, Ple I, Sap I, SfaN I, Sml I, Sfi I, TspR I, Tth111 I or Xcm I and the like sold by NEB company, or some special restriction endonucleases (nick enzyme), such as Nb.Bsm I, Nb.BsrD I, Nb.bts I, Nt.Alw I and Nt.BstNB I and the like, they also can enzymatically cleave to produce unself-linked cohesive ends. In view of comprehensive consideration about multiple factors, the restriction endonuclease which can recognize 6 basic bases and enzymatically cleave to produce unself-linked cohesive ends with more than 1 basic base is preferred, such as AlwN, Bbs I, Bgl I, Blp I, Bsa I, BsmB I, BspM I, BsrD I, BstAP I, BstE II, BstX I, Bsu36 I, Bts I, Dra III, Drd I, Ear I, Eci I, PflM I, Sap I or Sfi I and the like. Among these enzymes, the restriction endonuclease which can selectively recognize 6 basic bases and enzymatically cleave to produce cohesive ends with more than 3 basic bases is more preferred, such as Bbs I, Bsa I, BsmB I, BspM I, BstE II or BstX I and the like.

The invention further provides another DNA molecule for expressing hairpin RNA (inverted repeat structure double-strand DNA molecule). One strand of the DNA molecule is said DNA molecule for expressing hairpin RNA, another strand is complementary to the DNA molecule for expressing hairpin RNA.

Said DNA molecule for expressing hairpin RNA can be made with the method as follows:

Said DNA molecule for expressing hairpin RNA (closed single-strand cyclic molecule) is used as a template to synthesize the DNA molecule for expressing hairpin RNA (inverted repeat structure double-strand DNA molecule) under the action of DNA polymerase with the strand displacement ability.

Wherein, there are various DNA polymerases, such as Bst DNA polymerase, Vent DNA polymerase and Klenow DNA polymerase and the like, and more specifically, Phi29 DNA polymerase.

The closed single-strand cyclic DNA molecule is amplified by a rolling circle replication mode under the action of Phi29 DNA polymerase to synthesize a great amount of double-strand DNA molecules with inverted repeat structure, and this method using the rolling circle replication mode to obtain DNA with inverted repeat structure is known as RMHR system, i.e. rolling-cycle amplification (RCA)-mediated hairpin RNA system (RMHR).

A further object of the invention is to provide a constructing method for a gene silencing vector.

The constructing method for the gene silencing vector of the invention comprises the steps as follows:

1) Said DNA molecule for expressing hairpin RNA (closed single-strand cyclic DNA) is constructed with said constructing method for the DNA molecule for expressing hairpin RNA (closed single-strand cyclic DNA);

2) Said DNA molecule for expressing hairpin RNA (inverted repeat structure double-strand DNA molecule) is constructed with said constructing method for the DNA molecule for expressing hairpin RNA (inverted repeat structure double-strand DNA molecule);

3) Said DNA molecule for expressing hairpin RNA (inverted repeat structure double-strand DNA molecule) is enzymatically cleaved by the restriction endonucleases to obtain a DNA fragment encoding the hairpin RNA, and said restriction endonucleases meet the requirement of □ or □ as follows:

□ recognizing said recognition sequence R;

□ recognizing said recognition sequence S;

4) The DNA fragment encoding the hairpin RNA obtained in step 3) is inserted into the multiple cloning site of the gene expression vector to obtain a gene silencing vector.

The recombinant bacteria or transgenic cell lines containing said DNA molecule for expressing hairpin RNA (inverted repeat structure double-strand DNA molecule) of the invention are also within the scope of the invention.

### BRIEF DESCRIPTION OF DRAWINGS

FIG. 1 is a schematic view for constructing the hpRNA silencing vector.

FIG. 2 is the enzyme cleavage identification result of GUS gene linking to pBsa vector.

FIG. 3 is the secondary structure map of the DNA fragment with the stem-loop structure: miniHP1 and miniHP2.

FIG. 4 is the identification result of the inverted repeat sequence of GUS gene (hpGUS).

FIG. 5 depicts the hpGUS silencing to the GUS gene.

FIG. 6 is the secondary structure map of the DNA fragment with the stem-loop structure: intronHP.

FIG. 7 is the enzyme cleavage identification of the gene silencing library.

FIG. 8 is the secondary structure map of DNA fragment with the stem-loop structure: T hairpin.

### BEST MODES FOR CARRYING OUT THE INVENTION

The method of constructing the DNA molecule for expressing hairpin RNA (inverted repeat structure double-strand DNA molecule) of the invention comprises the steps as follows:

(1) The hairpin DNA with asymmetric cohesive ends is designed and synthesized, the cohesive ends have to avoid the hairpin DNA to self link or avoid two different hairpin DNA to link, and the enzyme cleavage sites of restriction endonuclease are introduced into the stem or loop of the hairpin DNA in order to identify and clone the final target DNA fragment;

(2) Two ends of the target DNA fragment to be silenced are treated to be the unself-linked asymmetric cohesive ends (such as using the specific restriction endonuclease or Taq enzyme); the cohesive ends can be linked to the cohesive ends of the hairpin DNA in (1);

(3) The DNA fragments in (1) and (2) are linked to be a closed single-strand cyclic DNA molecule;

(4) The closed single-strand cyclic DNA molecule in step (3) is synthesized to the double-strand DNA molecule under the action of the DNA polymerase to form the double-strand DNA molecule with inverted repeat structure, wherein the DNA polymerase is preferentially selected from the DNA polymerase with the rolling circle replication function (such as DNA polymerase Phi29), which not only can be synthesized to the double-strand DNA, also can make the closed single-strand cyclic DNA molecule to significantly amplify.

The DNA molecule for expressing hairpin RNA of the invention (inverted repeat structure double-strand DNA molecule) can be used to construct the gene silencing vector, the double-strand DNA molecule with inverted repeat structure in step (4) above is enzymatically cleaved by enzyme and links to the expression vector to construct the gene silencing vector on the basis of obtaining the DNA molecule for expressing hairpin RNA (inverted repeat structure double-strand DNA molecule).

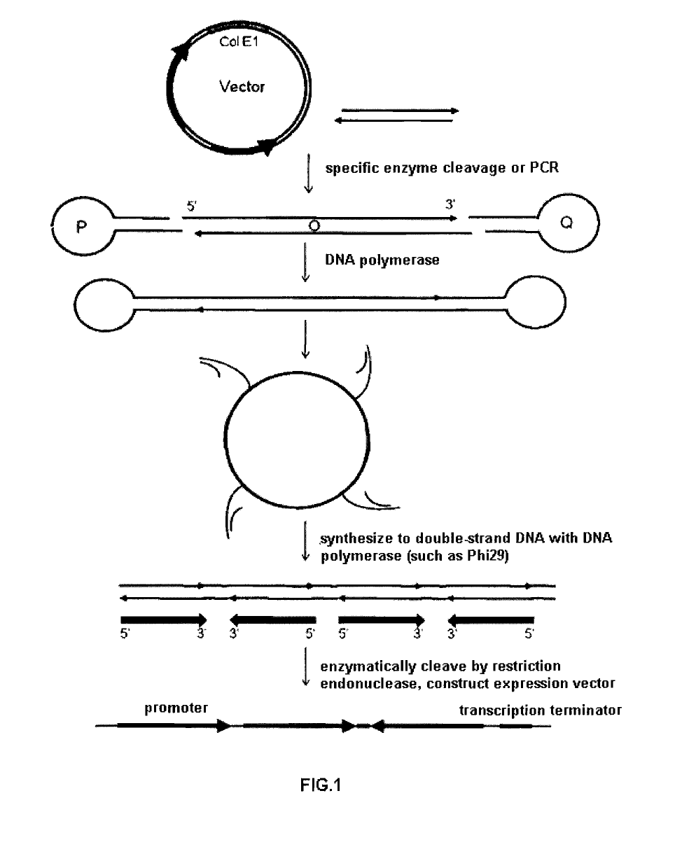

The following examples are provided to understand the invention better, but not to limit the invention. FIG. 1 is a schematic view for constructing the hpRNA silencing vector.

### Example 1

### Construction of the pBI121HPGUS Vector for Silencing GUS Gene

1. Construction of the Cloning Vectors Containing Three Restriction Endonuclease Recognition Sequences a, b and c

Two DNA chains Bsa(+) and Bsa(−) are synthesized, the sequences of Bsa(+) and Bsa(−) are as follows:

Bsa(+):
(SEQ ID NO: 5 in the sequence list)
5′ GGTCTCTGGGATATCGGTCTCTGAAG 3′;
Bsa(−):
(SEQ ID NO: 5 in the sequence list)
5′ CTTCAGAGACCGATATCCCAGAGACC 3′;

Two chains Bsa(+) and Bsa(−) are annealed to form double-strand Bsa fragment. The annealing system is: Bsa(+) 50 μM, Bsa(−) 50 μM 1 μL, 10×PCR buffer 5 μL, MgCl₂ □2.5 mM□3 μL, made up to the final volume of 50 μL with water. The annealing conditions are: 88□, 2 min; 65□, 10 min; 37□, 10 min; 25□, 5 min.

Two DNA chains BsaT(+) and BsaT(−) are synthesized, the sequences of BsaT(+) and BsaT(−) are as follows:

BsaT(+):
(SEQ ID NO: 7 in the sequence list)
5′GGTCTCTGGGATATCCAAGCTTTAAATGGTTATAGCCATGGAACTAG
TGGATCCCGGGTCTCTGAAG 3′;
BsaT(−):
(SEQ ID NO: 8 in the sequence list)
5′CTTCAGAGACCCGGGATCCACTAGTTCCATGGCTATAACCATTTAAA
GCTTGGATATCCCAGAGACC 3′;

Two chains BsaT(+) and BsaT(−) are annealed to form double-strand BsaT fragment, the annealing method is the same as above.

The fragments Bsa and BsaT are inserted into the vector pUC18 at SmaI site to obtain the vectors pBsa and pBsa-T respectively. The vector pBsa-T contains Bsa I and Xcm I sites, and the enzyme cleavage of Xcm I can produce a DNA vector with 3′ cohesive ends being “T”, which is convenience for inserting the PCR products.

2. The Double-Strand DNA Fragment of GUS Gene is Cloned into the pBsa Vector.

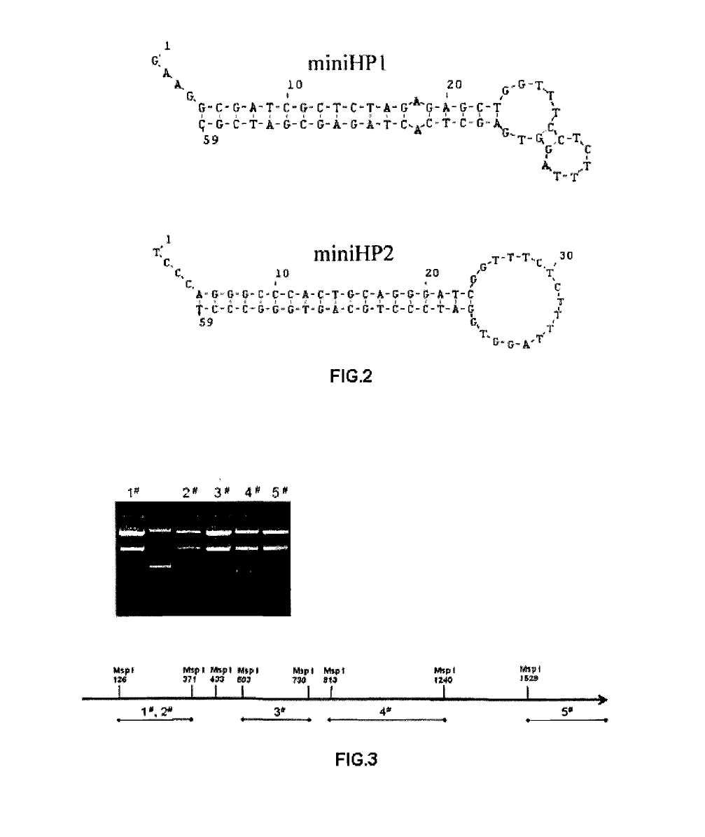

The vector pBI121 is enzymatically cleaved by enzymes BamH I and Sac I, 0.5 μg 1.87 kb GUS gene fragment is isolated and purified, then is enzymatically cleaved by enzyme Msp I, 0.1 μg 200-600 bp fragment is recovered via agarose gel electrophoresis and end filling is carried out with T4 DNA polymerase according to the instruction, then it is inserted into the vector pBsa at EcoR V site, E. coli is transformed to obtain more than 4000 clones, 10 clones is selected randomly, identified with enzyme cleavage, among others 5 clones contained the inserted target fragment, the size is 200-400 bp, and the result of sequencing shows that they are evenly distributed in GUS gene from 5′ to 3′ end, as shown in FIG. 2. Wherein, the GUS gene fragment contained in pBsa-GUSF1# is 245 bp and the nucleotide sequence thereof is 126-371 bp region of GUS gene.

3. The Double-Strand DNA Molecule with Inverted Repeat Structure

a) Construction of the Closed Single-Strand Cyclic DNA Molecule

The DNA fragments with stem-loop structure—miniHP1 and miniHP2 are artificially synthesized and the nucleotide sequences of miniHP1 and miniHP2 are as follows:

miniHP1:
(SEQ ID NO: 1 in the sequence list)
5′-GAAGGCGATCGCTCTAGAGAGCTGGTTTCCTCTTTAGGTGAGCTCA
CTAGAGCGATCGC-3′;
miniHP2:
(SEQ ID NO: 2 in the sequence list)
5′-TCCCAGGGCCCACTGCAGGGATCGGTTTCTCTTTTAGGTGGATCCA
TGCAGTGGGCCCT-3′.

miniHP1 and miniHP2 each 1 μg are annealed respectively to form the hairpin DNA, the annealing method is the same as 1. The phosphorylation is carried out according to the instruction of T4 polynucleotide kinase, 1/10 volume of 3M NaOAc and 2.5 times volume of ethanol are added to the product of phosphorylation to precipitate, the precipitate is dried and then dissolve in the sterile water to form the DNA fragment with stem-loop structure, the DNA fragment contains the cohesive ends preventing self-linkage (FIG. 3).

The pBsa-GUSF1# is enzymatically cleaved by Bsa I and electrophoresed, and about 300 bp GUS gene fragment is recovered for the linking reaction. The linking reaction system is: about 10 ng 300 bp GUS gene fragment, 10 ng miniHP1, 10 ng miniHP2, 2 μL 10×T4 DNA ligase buffer and 0.5 μL T4 DNA ligase are added into 20 μl linking system. The linking reaction is carried out overnight at 16° C., then 1/10 volume of 3M NaOAc and 2.5 times volume of ethanol are added in to precipitate. The precipitate is dried and then dissolved in 204 sterile water to form the closed single-strand cyclic DNA solution.

b) Construction of the Double-Strand DNA Molecule with Inverted Repeat Structure

The closed single-strand cyclic DNA in a) is converted into the double-strand DNA molecule using DNA polymerase Phi29 with the ability of strand displacement to form the double-strand DNA molecule with inverted repeat structure.

The reaction system is as follows: 2 μL 10×phi29 DNA polymerase buffer (New England Biolabs) and 1 μL 100 μM 6 nt random primer are added into 24 the closed single-strand cyclic DNA solution above; the mixture above is heated at 95□ for 3 min and placed onto the ice for 10-15 min, then 0.5 μL 10 mM dNTP, 0.2 μL 10 mg/mL BSA and 0.5 μL phi29 DNA polymerase (New England Biolabs) are added in, and water is added to the final volume of 20 μL. The reaction is carried out at 30□ for over 4 hrs, then 1/10 volume of 3M NaOAc and 2.5 times volume of ethanol are added in to precipitate. The precipitate is dried and then dissolved in 20 μL sterile water to form the solution containing the double-strand cyclic DNA with inverted repeat structure.

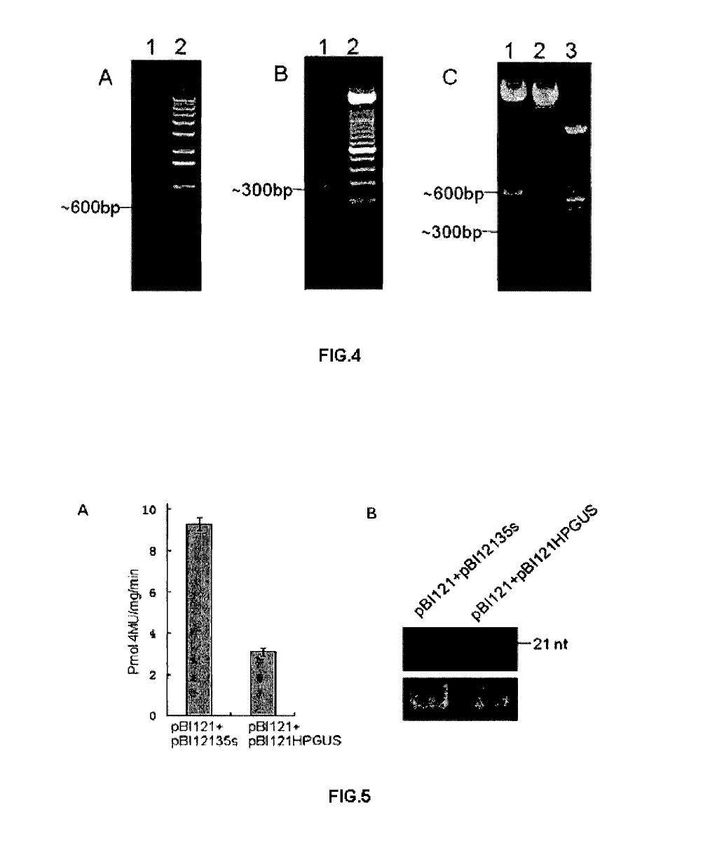

2 μL double-strand DNA molecule solution above is enzymatically cleaved by Xba I, electrophoresis is carried out after 1 hr, about 600 bp DNA fragment is observed, the product of enzyme cleavage is further enzymatically cleaved by BamH I and electrophoresis is carried out to reveal the DNA fragment of about 300 bp, whilst the band of 600 bp disappears, as shown in FIGS. 4A and 4B. In FIG. 4A, “1” represents the double-strand DNA after Xba I enzyme cleavage and amplification and “2” represents Marker; in FIG. 4B, “1” represents 600 bp DNA fragment of BamH I enzyme cleavage and “2” represents Marker. Because Xba 1 is located in miniHP1 and BamH I is located in miniHP2, the result above shows that the single-strand cyclic DNA is effectively amplified, the DNA fragment with inverted repeat structure is produced and the DNA can use to express hairpin RNA. The system based on Phi29 DNA polymerase rolling-cycle amplification method to amplify the single-strand cyclic DNA and obtain the DNA with inverted repeat structure is termed rolling-cycle mediated hairpin RNA system (RMHR system, rolling-cycle amplification (RCA)-mediated hairpin RNA system).

4. The Construction of pBI121HPGUS Vector

The product of amplification above is enzymatically cleaved by Xba 1 and Sac I, about 600 bp DNA fragment is isolated and purified through the agarose electrophoresis, pBI121 vector is also enzymatically cleaved by Xba I and Sac I and about 11 kb fragment of the vector is recovered. The enzyme-link system is as follows: 20 ng 600 bp DNA, 10 ng pBI121 vector, 1 μL 10×T4 polymerase buffer, 0.5 μL T4 DNA ligase, and water is added to 10 μL, the link reaction is carried out overnight at 16□, E. coli is transformed, the correct clone is identified with enzyme cleavage, and GUS gene silencing vector pBI121HPGUS is obtained; the result of enzyme cleavage identification of pBI121HPGUS is shown in FIG. 4C. In FIG. 4C, “1” represents pBI121HPGUS vector with Xba 1 and Sac I enzyme cleavage, and about 600 bp fragment is enzymatically cleaved from pBI121HPGUS vector; “2” represents pBI121HPGUS vector with three enzymes Sac I, BamH I and Xba I enzyme cleavage, and about 300 bp fragment is enzymatically cleaved from pBI121HPGUS, which indicate that the sequence inserted has inverted repeat structure, and the result is further confirmed through DNA sequencing.

5. The Effect of pBI121HPGUS Silencing Vector on the Expression of GUS Gene

pBI121HPGUS and pBI121 are transferred into Agrobacterium GV310 respectively, the pBI121 vector is self linked after enzymatically cleaved by Sma I and Ecl136II to obtain the vector pBI121-35S, and the vector pBI121-35S is also transferred into GV310 as control. Nicotiana benthamiana grows at about 24□ and 16 h light/8 h dark, and uses to perform Agrobacterium injection experiment when it grows to 8-10 leaves. The method of Agrobacterium injection experiment is as follows: the single Agrobacterium clones containing pBI121HPGUS, pBI121 and pBI121-35S are picked, inoculated into 3 mL YEB medium (with 50 mg/L rifamycin and 50 mg/L kanamycin), incubated overnight at 28° C. and then transferred into 15 ml YEB medium (with 50 mg/L rifamycin, 50 mg/L kanamycin, 10 mM MES and 20 mM acetosyringone), and incubated at 28° C. for 8 hrs, then the thallus is collected and resuspended in the injection buffer (10 mM MgCl₂, 10 mM MES and 200 mM acetosyringone), OD₆₀₀ is adjusted to 2.0, it is settled at the room temperature for 2-3 hrs. 4 mL pBI121HPGUS Agrobacterium and 1 mL pBI121 Agrobacterium are mixed uniformly, and injected into the leaves of Nicotiana benthamiana with 1 mL disposable injector. pBI121-35S and pBI121 Agrobacterium are mixed uniformly and injected into Nicotiana benthamiana as control by the same method as above. The injected plants grow under the same culture condition for other 48 hrs, and the GUS activity quantitative analysis and Northern blot hybridization are performed.

The method of the GUS activity quantitative analysis is as follows:

50 mg tobacco's leaves injected Agrobacterium are milled to powder with liquid nitrogen, 0.5 mL protein extracting buffer is added (50 mM Na₂H₂PO₄, 10 mM EDTA, 0.1% Triton X-100 and 1.0 g L⁻¹ lauroyl sarcosine sodium), they are centrifuged at 10000 g for 5 min after mixing uniformly, the supernatant is recovered for GUS activity measurement. The measurement of the total protein concentration is referred to Bradford's method (Bradford, M. M. (1976) Anal Biochem 72, 248-254). The measurement of GUS activity is performed using MUG as substrate; the specific measurement is referred to the article of Jefferson et. al. (Jefferson, R. A., EMBO J. 6, 3901-3907). The apparatus for measurement is Hoefer DyNA Quant 200 from Amersham Biosciences Company, and the operational procedure is performed according to the operating instruction provided.

The result of GUS activity quantitative analysis is shown in 5A, indicating that the GUS activity in the leaves injected pBI121HPGUS and pBI121 is significantly lower than the GUS activity in the leaves injected pBI121-35S and pBI121.

The method of Northern blot hybridization is as follows: 100 mg tobacco's leaves injected Agrobacterium is milled to powder with liquid nitrogen, 1 mL Trizol (Invitrogen) is added, the total RNA is exacted and the operational procedure is performed according to the instruction. The isolation, electrophoresis and transferring to a membrane of the total RNA are carried out according to Wang's article (Wang, L., FEBS Lett 581, 3848-3856). pBsa-GUSF1# plasmid is enzymatically cleaved by Bsa I and about 300 bp GUS DNA fragment is recovered as probe, the labeling kit is Prime-a-Gene Labeling System (Promega), ³²P-dCTP label. The method of hybridization is performed according to the article of Wang L. et. al. (Wang, L., FEBS Lett 581, 3848-3856). The sign of hybridization is detected using the autoradiography.

The result of Northern blot hybridization is shown in FIG. 5B, there is about 22 nt siRNA band in the RNA of the leaves injected pBI121HPGUS and pBI121, whereas there is no siRNA sign in the RNA of the leaves injected pBI121-35S and pBI121.

The results of the experiments above indicate that the silencing vector pBI121 HPGUS constructed can produce siRNA silencing GUS gene, which silence the GUS gene expressed by pBI121 vector.

### Example 2

### The Construction of Arabidopsis Hairpin RNA Silencing Library

1. The Construction of Arabidopsis cDNA Library Based on pBsa-T Vector

The Arabidopsis cDNA library is purchased from ABRC (Arabidopsis Biological Resource Center, Stock Number CD4-7) and the amplification of the library and the extracting method of the plasmid are referred to the instruction provided by ABRC.

10 μg library's cDNA is extracted, the cDNA fragment is enzymatically cleaved from the vector by Not I and Sal I, about 1 μg 400-2000 bp DNA fragment is recovered, then the DNA fragment recovered is enzymatically cleaved by Alu I, the product of enzyme cleavage is precipitated with 1/10 volume of 3M NaOAC and 2.5 times volume of ethanol, the precipitate is dried and dissolved in 20 μl sterile water; 3 μl 10×PCR buffer, 0.5 μl 10 mM dATP and 1 U Taq are added, and water is added to 30 μL, they react at 72□ for 30 min, “A” are added at the 3′ end of the product of enzyme cleavage. About 100 ng 100-1000 bp Arabidopsis cDNA fragment is recovered through the agarose electrophoresis.

pBsa-T vector is enzymatically cleaved by Xcm I and the pBsa-T vector obtained is used to perform the linking reaction, the system of linking reaction is as follows: 20 ng Arabidopsis cDNA fragment recovered, 10 ng pBsa-T vector, 2 μL 10×T4 DNA ligase, 0.5 μL ligase, and water is added to 20 μL. The linking reaction is carried out at 16° C. overnight. The product of linkage is used to transform E. coli to obtain Arabidopsis cDNA library pBsa-Atlib containing 10⁶ clones. The library obtained is amplified, and the method of amplification is performed according to the instruction of SuperScript™ Plasmid System for cDNA Synthesis and Plasmid Cloning Kit from GibcoBRL Company. The plasmid DNA is exacted from the library amplified, 3 μg DNA is taken and enzymatically cleaved by Bsa I, and about 100 ng 100-1000 bp DNA fragment is recovered through the agarose electrophoresis.

2. Obtaining the Closed Single-Strand Cyclic Arabidopsis cDNA

The intronHP DNA sequence with the intron is designed and synthesized in order to improve the silencing efficiency of gene and enhance the stability of the inverted repeat sequence in bacteria, and the sequence of intronHP is as follows: 5′TCCCGGTGCTCTCGCGACAGCTGTTGATCAATGTTTTTGTGTTTCTGAGTCTTCA GACTTTGCAATATGCAGAGCTGTCGCGAGAGCACC3′(SEQ ID NO:3 in the sequence list), the DNA strand is self-annealed to form the hairpin DNA structure with the intron (FIG. 6), and the method is the same as the step 3 of Example 1.

10 ng 100-1000 bp DNA fragment recovered is taken and added with 10 ng miniHP1 or 10 ng intronHP and 2 μl 10×T4 ligase reaction buffer and 0.5 μl T4 DNA ligase, then water is added to 20 μL, the linking reaction is carried out at 16□ overnight, then 1/10 volume of 3M NaOAC and 2.5 times volume of ethanol are added to precipitate, the precipitate is dried and dissolved in 20 μl sterile water to obtain the closed single-strand cyclic DNA.

3. Construction of the Double-Strand DNA Molecule with Inverted Repeat Structure

The single-strand cyclic DNA is amplified with Phi29 DNA polymerase and the reaction system is as follows: 2 μL single-strand cyclic DNA solution above is taken, 2 μL 10×Phi29 DNA polymerase buffer and 1 μL 100 μM 6 nt random primer are added in; and the mixture as above is heated at 95° C. for 3 mins, and then placed on the ice for 10-15 mins, 0.5 μL 10 mM dNTP, 0.2 μL 10 mg/mL BSA and 0.5 μL phi29 DNA polymerase are added in, and water is added to the final volume of 20 μL.

The reaction system above is reacted at 30° C. for more than 4 hrs, then added 1/10 volume of 3M NaOAC and 2.5 times volume of ethanol to precipitate, the precipitate is dried and dissolved in 30 μl sterile water to obtain Arabidopsis's double-strand DNA with inverted repeat structure.

4. Obtaining Arabidopsis Hairpin RNA Silencing Library pBI121-hpRNA

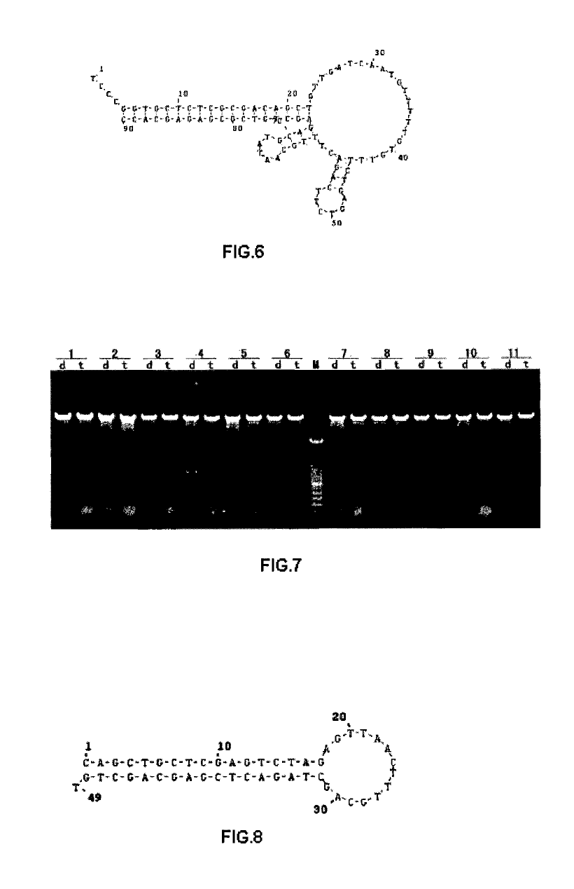

4 μL 10×enzyme cleavage buffer, 1 μL Xba 1 and 1 μL Sac I are added into the double-strand DNA solution above, water is added to 40 μL, the enzyme cleavage reaction is performed at 37° C. for 2 hrs, 200-2000 bp DNA fragment is recovered though electrophoresis and links with pBI121 vector which is enzymatically cleaved by Xba I and Sac I and recovered to obtain the recombinant plasmid pBI121-hpRNA, pBI121-hpRNA is used to transform E. coli to get 10⁵ clones, 12 clones are randomly selected and identified with Xba I and Sac I enzyme cleavage, wherein 11 clones are the recombinant plasmid pBI121-hpRNA, indicating that above 90% clones of the library constructed have inserted the target DNA fragment, pBI121-cDNA is enzymatically cleaved by Xba I, Sac I and Pvu II and the inserted fragments are reduced half, indicating that the inserted fragments are the inverted repeat structure, as shown in FIG. 7. In FIG. 7, “d” represents Xba I and Sac I enzyme cleavage; “t” represents Xba I, Sac I and PvuII enzyme cleavage, “1-11” represents 11 clones containing pBI121-hpDNA and “M” represents Marker. 20 single clones are randomly picked and experienced. DNA sequencing, the result of sequencing is shown that above 95% sequences of the hairpin library constructed come from Arabidopsis's cDNA and have the inverted repeat structure, the product expressed can form the hairpin RNA. The results above indicate that the hairpin RNA silencing/RNAi library of Arabidopsis is successfully constructed using the RMHR method above (the step 3 of Example 1).

### Example 3

### Constructing Hairpin RNA Vector with PCR Product

1. Obtaining Short Hairpin DNA

The single-strand DNA-T hairpin is artificially synthesized and the nucleotide sequence thereof is as follows:

T hairpin: 5′ cagctgctcgagtctagagttaaCtttgcagCTAGACTCGAGCAGCTGt 3′ (SEQ ID NO:4 in the sequence list).

The sequences are annealed to form the hairpin DNA with stem-loop structure, the method is the same as the step 3 of Example 1, and 3′ end of stem of the hairpin DNA is cohesive end with “T” (FIG. 8).

2. Obtaining the PCR Product

The primers GUSfw and GUSry are synthesized and their sequences are as follows:

GUSfw:
(SEQ ID NO: 9 in the sequence list)
5′ AAGCTTCGGGCAATTGCTGTGCCAG3′;
GUSrv:
(SEQ ID NO: 10 in the sequence list)
5′ CGGCAATAACATACGGCGTG3′.

The GUS gene is amplified using GUSfw and GUSrv.

PCR system: 1 ng pBI121, 5 μL 10×buffer, 10 μM GUSfw and GUSrv each 2 μL, 1 μL 10 mM dNTP, 1 U Pfu, and water is added to 504.

PCR condition: 94° C. 3 min, 94° C. 30 sec, 55° C. 30 sec, 72° C. 30 sec, 30 cycles, to obtain 246 bp DNA fragment of 127-373 bp of GUS gene.

1/10 volume of 3M NaOAC and 2.5 times volume of ethanol are added in the PCR product to precipitate, the precipitate is dried and dissolved in 204 sterile water; 3 μL 10×buffer and 0.5 μL 10 mM dATP, 1 U Taq are added in, then water is added to 30 μL, reaction is carried out at 72° C. for 30 min to add “A” at 3′ end of the PCR product. The product is experienced the agarose electrophoresis to recover about 250 bp DNA fragment.

3. Obtaining the Closed Single-Strand Cyclic DNA

About 20 ng 250 bp GUS gene fragment, 10 ng T hairpin, 2 μL 10×T4 ligase reaction buffer, 0.5 μL T4 ligase are added into 20 μL linking system. The linking reaction is carried out at 16° C. overnight, 1/10 volume of 3M NaOAC and 2.5 times volume of ethanol are added in the product to precipitate, the precipitate is dried and dissolved in 2 μL sterile water to obtain the closed single-strand cyclic DNA solution.

4. Construction of the Double-Strand DNA Molecule with Inverted Repeat Structure

The single-strand cyclic DNA obtained above is amplified with Phi29 DNA ligase, and the amplification method and the reaction system are the same as Example 1. The product amplified is experienced the agarose electrophoresis, and the large fragment of DNA product with inverted repeat structure is observed, the product is enzymatically cleaved by HindIII to produce about 550 bp DNA fragment, the fragment may be further enzymatically cleaved by Xba I to produce about 250 bp DNA fragment, and the results above indicate that the double-strand DNA molecule with inverted repeat structure is obtained using the RMHR method as above (the step 3 in Example 1)

### INDUSTRY APPLICATION

The invention provides a DNA molecule for expressing hairpin RNA, a constructing method and application thereof. The invention firstly constructs a closed single-strand cyclic DNA molecule, the DNA molecule is used as the template, the closed single-strand cyclic DNA molecule is amplified using DNA polymerase such as Phi29 to form the double-strand DNA molecule with inverted repeat structure, the double-strand DNA molecule can produce DNA with inverted repeat structure only with suitable enzyme cleavage, the DNA can produce RNA with hairpin structure after transcription, thus the method has the characteristics of rapidness, high-throughput and low cost, and can be used to construct the gene silencing vector with hairpin structure with various length to silence one and more target genes. The long hairpin RNA (lhRNA) library has the application potential in exploiting and identifying gene function and the like areas, there is still no report about the construction method for long hairpin RNA (lhRNA) whose length of stem is above 40 bp, the construction method for the DNA molecule for expressing hairpin RNA provided by the invention has the function which can not be replaced in constructing the long hairpin RNA (lhRNA) library, the DNA from various biologic sources such as animals, plants and microorganisms can construct the gene silencing/RNAi vector or library.

1. A DNA molecule for expressing hairpin of the invention is a closed single-strand cyclic molecule consisting of four single-strand DNA fragments A, B, C and D; and said four fragments A, B, C and D are linked in the order of 1) or 2) as follows:
1) A-C-B-D;
2) A-D-B-C;
A is a fragment selected from a sense or a antisense strand of the target double-strand DNA fragment, and the size of said A is 40-3000 nt;
B is a fragment which is reversely complementary to A;
C and D are DNA fragments with the stem-loop structure, and said C and D fragments meet the requirement of 3) or 4) as follows:
3) The nucleotide sequence of said fragment C or D comprises at least one sequence e, said sequence e forms a recognition sequence R of restriction endonuclease with the complementary sequence thereof;
4) The nucleotide sequence of said fragment C comprises at least one sequence e, said sequence e forms a recognition sequence R of restriction endonuclease with the complementary sequence thereof; the nucleotide sequence of said fragment D comprises at least one sequence f, said sequence f forms a recognition sequence S of restriction endonuclease with the complementary sequence thereof; said recognition sequences R and S are recognized by the different restriction endonucleases.

2. The DNA molecule according to claim 1, wherein the size of said A is 100-2000 nt, 100-1000 nt, 200-800 nt or 200-600 nt.

3. The DNA molecule according to claim 1, wherein the nucleotide sequences of said C and D are SEQ ID NO:1 and SEQ ID NO:2 in the sequence list, respectively.

4. The DNA molecule according to claim 1, wherein the nucleotide sequences of said C and D are SEQ ID NO:1 and SEQ ID NO:3 in the sequence list, respectively.

5. The DNA molecule according to claim 1, wherein the nucleotide sequences of said C and D are SEQ ID NO:4 in the sequence list.

6. A method for constructing the DNA molecule according to claim 1, wherein three DNA fragments O, P and Q are linked to a closed single-strand cyclic molecule;
said fragment O is a target double-strand DNA fragment with two cohesive ends which can not be self linked.
said fragments P and Q are DNA fragments with stem-loop structure having cohesive ends respectively, the cohesive ends of said fragments P and Q are matched with the two cohesive ends of said fragment O, and said fragments P and Q meet the requirement of i) or ii) as follows:
i) the nucleotide sequence of fragment P or Q comprises at least one sequence e, said sequence e forms a recognition sequence R of restriction endonuclease with the complemented sequence thereof;
ii) the nucleotide sequence of said fragment P comprises at least one sequence e, said sequence e forms a recognition sequence R of restriction endonuclease with the complemented sequence thereof; the nucleotide sequence of said fragment Q comprises at least one sequence f, said sequence f forms a recognition sequence S of restriction endonuclease with the complemented sequence thereof; said recognition sequence R and S are recognized by the different restriction endonucleases.

7. The method for constructing the DNA molecule according to claim 6, wherein said fragment O is obtained according to the method as follows:
1) The exogenous fragment is inserted into the poly clone sites of a gene cloning vector or gene expression vector to obtain a recombinant vector M; the nucleotide sequence of said exogenous fragment contains three recognition sequences a, b and c of the restriction endonuclease, said sequences a and c can produce unself-linked cohesive ends after the restriction endonuclease enzyme cleavage; the linking order of said three recognition sequence is a-b-c;
2) Said target double-strand DNA fragment is inserted into said b site of said recombinant vector M to get a recombinant vector N;
3) The recombinant vector N is enzymatically cleaved by the restrictive endonuclease recognizing said sequence a and c to get said fragment O.

8. The method for constructing the DNA molecule according to claim 6, wherein said fragment O is obtained according to the method as follows:
1) The exogenous fragment is inserted into the multiple cloning site in the gene cloning vector or gene expression vector to obtain a recombinant vector M; the nucleotide sequence of said exogenous fragment contains two recognition sequences a and c of the restriction endonuclease, said sequences a and c produce the unself-linked cohesive ends after the restriction endonuclease enzyme cleavage; there is a fragment b between said a and c, said fragment b contains two restriction endonuclease recognition sequences b-1 and b-2, two free basic bases of two overhung ends produced after the restriction endonuclease enzyme cleavage said sequences b-1 and b-2 are T;
2) The recombinant vector M is enzymatically cleaved by the restriction endonuclease recognizing said b-1 and b-2 to obtain a recombinant vector T;
3) The target double-strand DNA fragment is prepared, and two ends of the double-strand DNA fragment are overhung ends with free basic bases A;
4) The recombinant vector T of step 2) and the double-strand. DNA fragment of step 3) are linked to obtain a recombinant vector N;
5) The recombinant vector N is enzymatically cleaved by the restriction endonuclease recognizing said sequences a and c to obtain said fragment O.

9. The method for constructing the DNA molecule according to claim 6, wherein said fragment O is obtained according to the method as follows:
1) The exogenous fragment is inserted into the multiple cloning site of a gene cloning vector or gene expression vector to obtain a recombinant vector M; the nucleotide sequence of said exogenous fragment contains three recognition sequences a, b and c of the restriction endonuclease, said sequences a and c produce the unself-linked cohesive ends after the restriction endonuclease enzyme cleavage; the order of linking said three recognition sequences is a-b-c;
2) The recombinant vector M is enzymatically cleaved by the restriction endonuclease recognizing said sequence b to prepare vector T;
3) The target double-strand DNA fragment is prepared, and two ends of the double-strand DNA fragment are overhung ends with free basic bases A;
4) The vector T of step 2) and the double-strand DNA fragment of step 3) are linked to obtain a recombinant vector N;
5) The recombinant vector N is enzymatically cleaved by the restriction endonucleases recognizing said sequences a and c to obtain said fragment O.

10. The method for constructing the DNA molecule according to claim 7, wherein the sequences of said a and c produce the unself-linked cohesive ends with at least one overhung basic base after restriction endonucleases enzyme cleavage.

11. The method for constructing the DNA molecule according to claim 10, wherein the sequences of said a and c produce the unself-linked cohesive ends with at least three overhung basic bases after restriction endonucleases enzyme cleavage.

12. (canceled)

13. (canceled)

14. (canceled)

15. (canceled)

16. (canceled)

17. (canceled)

18. (canceled)

19. The method for constructing the DNA molecule according to claim 8, wherein the sequences of said a and c produce the unself-linked cohesive ends with at least one overhung basic base after restriction endonucleases enzyme cleavage.

20. The method for constructing the DNA molecule according to claim 9, wherein the sequences of said a and c produce the unself-linked cohesive ends with at least one overhung basic base after restriction endonucleases enzyme cleavage.
